(19) United States
(12) Patent Application Publication
(10) Pub. No.: US 2009/0012308 A1
(43) Pub. Date: Jan. 8, 2009

(54) PROCESS FOR THE MANUFACTURE OF BENZYLSULFONYLARENES

(75) Inventors: Mahmut Levent, Bloomfield, NJ (US); Panolil Raveendranath, (US); Sanjay Raveendranath, Monroe, NY (US); Vijay Raveendranath, Albany, NY (US); Girija Raveendranath, Monroe, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/135,350

(22) Filed: Jun. 9, 2008

Related U.S. Application Data
(60) Provisional application No. 60/934,513, filed on Jun. 13, 2007.

(51) Int. Cl. C07D 231/56; C07C 315/00

(52) U.S. Cl. 548/361.5; 568/30; 568/34

(57) ABSTRACT

The present invention provides a process for the manufacture of an arylmethyl-sulfonylarene, R₁CH₂SO₂R₂, wherein R₁ and R₂ are each independently an optionally substituted phenyl or naphthyl group which process comprises reacting an arylmethylhalide, R₁CH₂-Hal wherein R₁ is as defined hereinabove and Hal is Cl, Br or I with a sodium arylsulfinate R₂SO₂Na wherein R₂ is as defined hereinabove in the presence of a base optionally in the presence of a solvent.

Also provided is the use of the inventive process in the manufacture of a 3-arylsulfonylindazole 5-HT6 ligand.

### CROSS-REFERENCE TO RELATED APPLICATIONS

This application claims the benefit of U.S. Provisional Application No. 60/934,513, filed Jun. 13, 2007, the entire contents of which are incorporated herein by reference.

### FIELD

The present invention relates to a process for the preparation of an arylmethylsulfonylarene compound. The invention further relates to the use of this process in the manufacture of 5-hydroxytryptamine-6 (5-HT6) ligands.

### BACKGROUND OF THE INVENTION

Arylsulfonylindazoles are an important class of 5-hydroxytryptamine-6 (5-HT6) ligands useful in the treatment of central nervous system (CNS) disorders related to or affected by the 5-HT6 receptor, such as cognitive disorders or anxiety disorders. Novel 3-arylsulfonylindazole compounds and their use as 5-HT6 ligands are described in US 2004/0167122; US 2007/0037802; U.S. Pat. No. 6,727,246; U.S. Pat. No. 6,995,176; and US 2007/0054896, the contents each of which are incorporated herein by reference in their entirety. A key intermediate in the preparation of said 3-arylsulfonylindazole compounds is a benzylsulfonylarene compound such as a benzylsulfonylnaphthalene or a benzylsulfonylbenzene.

### SUMMARY

The present invention provides a process for the manufacture of an arylmethylsulfonylarene, R₁CH₂SO₂R₂, wherein R₁ and R₂ are each independently an optionally substituted phenyl or naphthyl group which process comprises reacting an arylmethylhalide, R₁CH₂-Hal wherein R₁ is as defined hereinabove and Hal is Cl, Br or I with a sodium arylsulfinate R₂SO₂Na wherein R₂ is as defined hereinabove in the presence of a base optionally in the presence of a solvent.

Also provided is the use of the inventive process in the manufacture of a 3-arylsulfonylindazole 5-HT6 ligand. In particular, the present invention provides a process for the manufacture of a 3-sulfonylindazole 5-HT6 ligand of formula IV

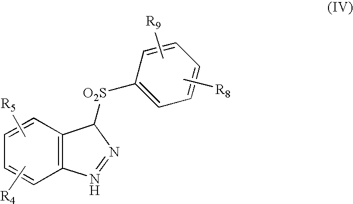

wherein R₄ and R₅ are each independently H, halogen, NO₂, NR₆R₇, an optionally substituted alkyl or an optionally substituted alkoxy group; R₆ and R₇ are taken together with the atom to which they are attached to form a 5- to 7-membered ring optionally containing an additional heteroatom selected from N, O or S; and R₈ and R₉ are each independently H, halogen, an optionally substituted alkyl or an optionally substituted alkoxy group or R₈ and R₉ when attached to adjacent carbon atoms are taken together with the atoms to which they are attached to form an optionally substituted 6-membered aromatic aryl ring

which comprises the following steps:

1) reacting a 2-nitrobenzylhalide of formula Ia

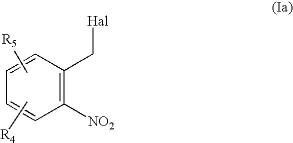

wherein R₄ and R₅ are as described hereinabove for formula IV and Hal is Cl, Br or I with a sodium arylsulfinate of formula II

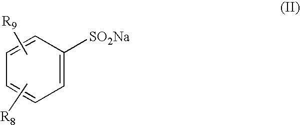

wherein R₈ and R₉ are as described hereinabove for formula IV in the presence of a base to give a compound of formula IIIa

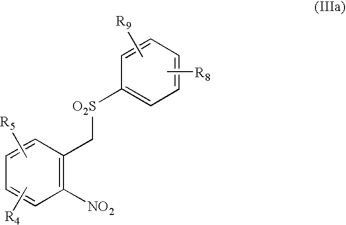

wherein R₄, R₅, R₈ and R₉ are as described hereinabove;

2) reacting said formula IIIa compound with reducing agent to give an amine of formula V

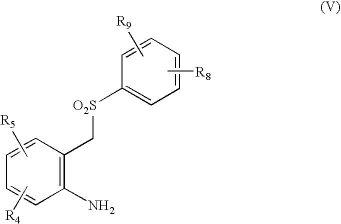

optionally in the presence of a solvent; and

3) reacting said formula V amine with NaNO₂ in the presence of an acid optionally in the presence of a solvent to give the desired 3-sulfonylindazole 5-HT6 ligand of formula IV.

The present invention also provides a process for the manufacture of a compound of formula IV described hereinabove

which comprises the following steps:

1) reacting a compound of formula Ib

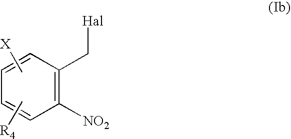

wherein X is either an activating group Gₐ or R₅; R₄ and R₅ are as described hereinabove for formula IV and Hal is Cl, Br or I with a compound of formula II

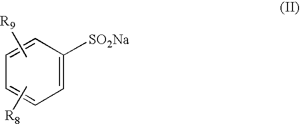

wherein R₈ and R₉ are as described hereinabove for formula IV in the presence of a base to give a compound of formula IIIb

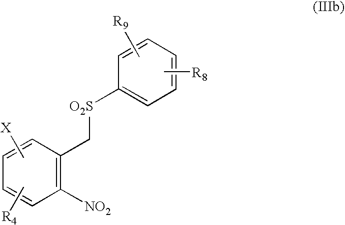

wherein X, R₄, R₈ and R₉ are as described hereinabove,

wherein, where X in formula Ib is R₅ and R₅ is NR₆R₇, the process optionally comprises reacting a compound of formula Ic

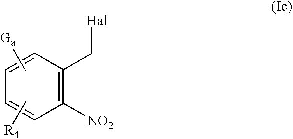

with HNR₆R₇ to give the compound of formula Ib;
with the proviso that if X of formula IIIb is Gₐ and R₅ of formula IV is NR₆R₇, then the process further comprises reacting said compound of formula IIIb with HNR₆R₇ to give the compound of formula IIIb wherein X is R₅ and R₅ is NR₆R₇;

2) reacting said compound of formula IIIb wherein X is R₅ with a reducing agent optionally in the presence of a solvent to give a compound of formula V

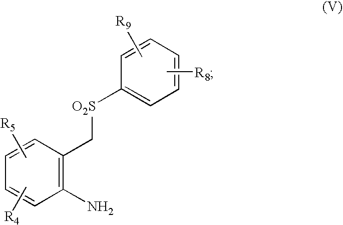

and

3) reacting said compound of formula V with NaNO₂ in the presence of an acid optionally in the presence of a solvent to give the compound of formula IV.

Further provided is a process of preparing a compound of formula IV described hereinabove, which process comprises reacting a compound of formula V

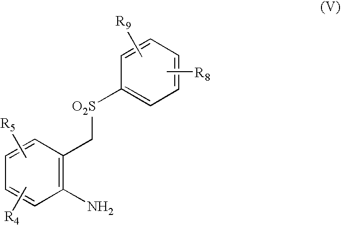

wherein R₄, R₅, R₈ and R₉ are as described above for formula IV with NaNO₂ in the presence of an acid optionally in the presence of a solvent to give the compound of formula IV.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION

The ability of the 5-hydroxytryptamine-6 (5-HT6) receptor to bind a wide range of therapeutic compounds used in psychiatry, coupled with its intriguing distribution in the brain has stimulated significant interest in compounds which are capable of interacting with or affecting said receptor. Compounds known to be 5-HT6 ligands include 3-arylsulfonylindazole compounds such as those described in US 2004/0167122; US 2007/0037802; U.S. Pat. No. 6,727,246; U.S. Pat. No. 6,995,176; and US 2007/0054896. A key intermediate in the preparation of said 3-arylsulfonylindazole compounds is a benzylsulfonylarene compound such as a benzylsulfonylnaphthalene or a benzylsulfonylbenzene. Heretofor, this intermediate was formed by the vicarious nucleophilic substitution of the appropriate nitroarene with a chloromethylsulfone, ClCH₂SO₂R, wherein R represents the desired aryl group. The chloromethylsulfone is prepared by reacting bromochloromethane with the appropriate sodium arylsulfinate. However, the process using bromochloromethane requires extreme temperatures of −45° C. or lower and special handling of the reagent, as bromochloromethane is a known mutagen and a suspected ozone-depleting agent.

Surprisingly, it has now been found that arylmethylsulfonylarene compounds, including benzylsulfonylarene compounds, may be prepared effectively and efficiently by reacting an arylmethylhalide with a sodium arylsulfinate in the presence of a base, optionally in the presence of a solvent. Accordingly, the present invention provides a process for the manufacture of an arylmethylsulfonylarene, R₁CH₂SO₂R₂, wherein R₁ and R₂ are each independently an optionally substituted phenyl or naphthyl group which process comprises reacting an arylmethylhalide, R₁CH₂-Hal wherein R₁ is as defined hereinabove and Hal is Cl, Br or I with a sodium arylsulfinate R₂SO₂Na wherein R₂ is as defined hereinabove in the presence of a base optionally in the presence of a solvent. Advantageously, the process of the invention eliminates the use of bromochloromethane and does not require extreme low temperatures.

Bases suitable for use in the process of the invention include alkali metal carbonates such as K₂CO₃, Na₂CO₃, or the like; alkali metal bicarbonates such as KHCO₃, NaHCO₃, or the like; or any base known to be suitable for use in conventional synthetic procedures of coupling a sulfinate with an alkyl halide, preferably an alkali metal carbonate, more preferably K₂CO₃.

Solvents suitable for use in the inventive process include ethers such as tetrahydrofuran; amides such as dimethyl formamide; esters such as ethyl acetate; aromatic hydrocarbons such as toluene; aprotic solvents such as acetonitrile; or the like; preferably tetrahydrofuran.

Temperatures suitable for use in the process of the invention include temperatures in the range of 0° C. to the boiling point of the solvent. It is understood that the reaction rate is directly related to the reaction temperature, i.e. the higher the temperature the faster the reaction rate and the shorter the reaction time. However, excessively high reaction temperatures may lead to lower yields and product purity due to the potential increase in undesired side reactions. In general, reaction temperatures of about 0° C. to 70° C., are suitable.

In actual practice, one equivalent of an arylmethylhalide is admixed with at least one equivalent of a base such as an alkali metal carbonate or bicarbonate, preferably K₂CO₃, optionally in the presence of a solvent such as tetrahydrofuran, dimethyl formamide, ethyl acetate, toluene or acetonitrile, preferably tetrahydrofuran, to form a reaction mixture; the mixture is cooled to about 0° to 10° C.; the cooled reaction mixture is treated with one equivalent of a sodium arylsulfinate and stirred at 600-70° C. until the reaction is complete.

In one embodiment of the invention, the process comprises reacting a benzylhalide of formula I

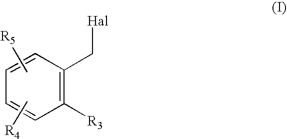

wherein R₃, R₄ and R₅ are each independently H, halogen, NO₂, NR₆R₇, an optionally substituted alkyl or an optionally substituted alkoxy group; R₆ and R₇ are taken together with the atom to which they are attached to form a 5- to 7-membered ring optionally containing an additional heteroatom selected from N, O or S; and Hal is Cl, Br or I with a sodium arylsulfinate of formula II

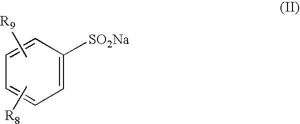

wherein R₈ and R₉ are each independently H, halogen, an optionally substituted alkyl or an optionally substituted alkoxy group or R₈ and R₉ when attached to adjacent carbon atoms are taken together with the atoms to which they are attached to form an optionally substituted 6-membered aromatic aryl ring in the presence of a base optionally in the presence of a solvent to give a benzylsulfonylarene of formula III

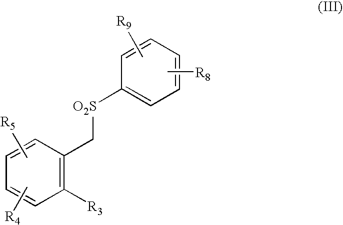

wherein R₃, R₄, R₅, R₈ and R₉ are as described hereinabove. The reaction is shown in flow diagram I.

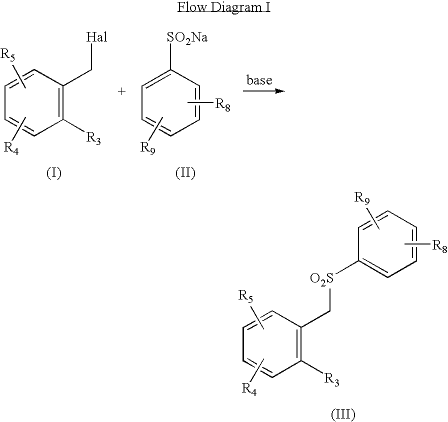

In the specification and claims, an optionally substituted moiety may be substituted with one or more substituents. The substituent groups, which are optionally present, may be one or more of those customarily employed in the development of pharmaceutical compounds or the modification of such compounds to influence their structure/activity, persistence, absorption, stability or other beneficial property. Specific examples of such substituents include halogen atoms, nitro, cyano, thiocyanato, cyanato, hydroxyl, alkyl, haloalkyl, alkoxy, haloalkoxy, amino, alkylamino, dialkylamino, formyl, alkoxycarbonyl, carboxyl, alkanoyl, alkylthio, alkylsuphinyl, alkylsulphonyl, carbamoyl, alkylamido, phenyl, phenoxy, benzyl, benzyloxy, heterocyclyl or cycloalkyl groups, preferably halogen atoms or lower alkyl or lower alkoxy groups. Unless otherwise specified, typically 0-4 substituents may be present. When any of the foregoing substituents represents or contains an alkyl substituent group, this may be linear or branched and may contain up to 12 carbon atoms, preferably up to 6 carbon atoms, more preferably up to 4 carbon atoms.

In another embodiment, the term “optionally substituted” means that the moiety is substituted with 0-4 substituents independently selected from halogen atoms, C₁-C₆alkyl, C₁-C₆alkoxy, C₁-C₆haloalkyl, C₁-C₆haloalkoxy, C₁-C₆alkylamino, di-C₁-C₆alkylamino or combinations thereof. In another preferred embodiment, the term “optionally substituted” means that the moiety is substituted with 0-4 substituents independently selected from halogen atoms, C₁-C₆alkyl, C₁-C₆alkoxy, C₁-C₆alkylamino, di-C₁-C₆alkylamino or combinations thereof. In another more preferred embodiment, the term “optionally substituted” means that the moiety is substituted with 0-4 substituents independently selected from halogen atoms, C₁-C₆alkyl, C₁-C₆alkylamino, di-C₁-C₆alkylamino or combinations thereof.

As used in the specification and claims, the ring “NR₆R₇” denotes an optionally substituted 5-7 membered heterocyclic ring. In one embodiment, “NR₆R₇” is an optionally substituted ring of formula VI:

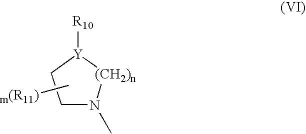

wherein m and n are each independently an integer of 1 to 3;
Y is CH or N with the proviso that if Y is N, n is 2 or 3; and
R₁₀ and each R₁₁ are independently selected from H, C₁-C₆alkyl, C₁-C₆alkoxy, C₁-C₆alkylamino or di-C₁-C₆alkylamino.

Reference to “activated” or “an activating group” or “Gₐ” as used herein is a group that, when bound to a center, increases the reactivity at that center. Non-limiting examples of an activating group include a substituent bound to an electrophilic center and capable of being displaced by a nucleophile; a substituent bound to a nucleophilic center and capable of being displaced by an electrophile; a substituent capable of being displaced by a radical; or a substituent bound to a center wherein, following gain or loss of an electron, the substituent is capable of leaving as an anion or cation with formation of a radical at the center. Examples of preferred activating groups are halogens, such as F, Cl, Br or I; triflate; mesylate, or tosylate; carbonyl groups in aldehydes or ketones; alkoxy groups in esters; the oxygen in epoxides; boronic acid groups (e.g. B(OH)₂); boronic ester groups, such as (B(Oalkyl)₂) and the like. An example of an activating group is chlorine in benzylchloride, which is readily attacked by a nucleophile, such as piperidine group to form a benzylpiperidine functionality.

As used herein, “activating” a compound refers to reacting the compound at a center with a reagent to introduce at the center an activating group, wherein the activating group is optionally converted to another activating group in one or more steps. Examples of activating include halogenation at a carbon center, optionally followed by hydroboration wherein the halogen group is converted to an optionally sustituted borane; tosylation, mesylation, or triflation at an oxygen center; and nitration at a carbon center optionally followed by reduction of the nitro group to an amino group and conversion of the amino group to a diazo group.

As used in the specification and claims, the term “aryl” designates a phenyl or naphthyl group.

As used herein, the term “alkyl” includes both a straight chain and a branched chain saturated hydrocarbon moiety. More particularly, “alkyl” refers to monovalent saturated aliphatic hydrocarbyl groups having from 1 to 12 carbon atoms and preferably, 1 to 6 carbon atoms (C₁-C₆alkyl) and more preferably, 1 to 4 carbon atoms (C₁-C₄alkyl). Examples of saturated hydrocarbon alkyl moieties, which are C₁-C₆alkyl groups include, but are not limited to, methyl (CH₃—); ethyl (CH₃CH₂—); propyl, e.g., n-propyl (CH₃CH₂CH₂—) and isopropyl ((CH₃)₂CH—); butyl, e.g., n-butyl (CH₃CH₂CH₂CH₂), tert-butyl ((CH₃)₃C—), isobutyl ((CH₃)₂CH₂CH₂—), and sec-butyl ((CH₃)(CH₃CH₂)CH—); pentyl, e.g., n-pentyl (CH₃CH₂CH₂CH₂CH₂—) and neopentyl ((CH₃)₃CCH₂—); and hexyl groups, e.g., n-hexyl (CH₃CH₂CH₂CH₂CH₂CH₂—), or the like. A branched alkyl group has at least 3 carbon atoms (e.g., an isopropyl group), and in various embodiments, has up to 6 carbon atoms. Examples of branched C₁-C₆alkyl groups include, but are not limited to:

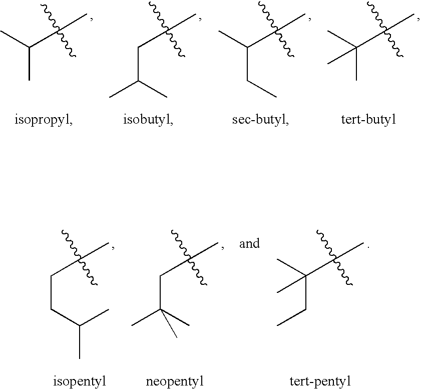

Specifically included within the definition of alkyl are those alkyl groups that are optionally substituted. Suitable preferred alkyl substitutions include, but are not limited to, CN, OH, halogen, amine, alkylamine, dialkylamine, phenyl, carbamoyl, carbonyl, alkoxy or aryloxy.

The term “alkoxy” as used herein, refers to the group alkyl-O— where alkyl group is as defined herein. Specifically included within the definition of alkoxy are those alkoxy groups that are optionally substituted. Suitable alkoxy substitutions include, but are not limited to, halogen, amine, alkylamine, dialkylamine, phenyl, carbamoyl, carbonyl or aryloxy, preferably dialkylamine.

“Amino” refers to the group —NH₂.

“Cyano” refers to the group —CN.

As used herein, the term “haloalkyl” designates a CₙH₂ₙ₊₁ group having from one to 2n+1 halogen atoms which may be the same or different. Examples of haloalkyl groups include CF₃, CH₂C₁, C₂H₃BrCl, C₃H₅F₂, or the like. A further example of a haloalkyl group is CHF₂.

The term “halogen” or “halo”, as used herein, designates fluorine, chlorine, bromine, and iodine.

“Hydroxy” or “hydroxyl” refers to the group —OH.

“Nitro” refers to the group —NO₂.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of another example, the term “5-7 membered ring” is specifically intended to individually disclose a ring having 5, 6, 7, 5-7, and 5-6 ring atoms.

Advantageously, the process of the invention may be used in the manufacture of a 3-sulfonylindazole 5-HT6 ligand. Accordingly, the present invention provides a process for the manufacture of a 3-sulfonylindazole 5-HT6 ligand of formula IV

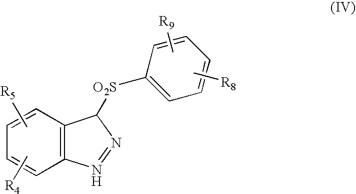

wherein R₄ and R₅ are each independently H, halogen, NO₂, NR₆R₇, an optionally substituted alkyl or an optionally substituted alkoxy group; R₆ and R₇ are taken together with the atom to which they are attached to form a 5- to 7-membered ring optionally containing an additional heteroatom selected from N, O or S; and R₈ and R₉ are each independently H, halogen, an optionally substituted alkyl or an optionally substituted alkoxy group or R₈ and R₉ when attached to adjacent carbon atoms are taken together with the atoms to which they are attached to form an optionally substituted 6-membered aromatic aryl ring

which process comprises the following steps:

1) reacting a 2-nitrobenzylhalide of formula Ia

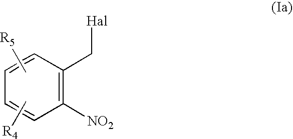

wherein R₄ and R₅ are as described hereinabove for formula IV and Hal is Cl, Br or I with a sodium arylsulfinate of formula II

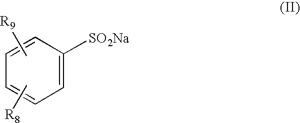

wherein R₈ and R₉ are as described hereinabove for formula IV in the presence of a base to give a compound of formula IIIa

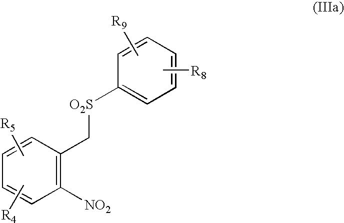

wherein R₄, R₅, R₈ and R₉ are as described hereinabove;

2) reacting said formula IIIa compound with reducing agent to give an amine of formula V

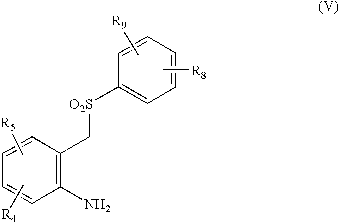

optionally in the presence of a solvent; and

3) reacting said formula V amine with NaNO₂ in the presence of an acid optionally in the presence of a solvent to give the desired 3-sulfonylindazole 5-HT6 ligand of formula IV.

The process is shown in flow diagram II.

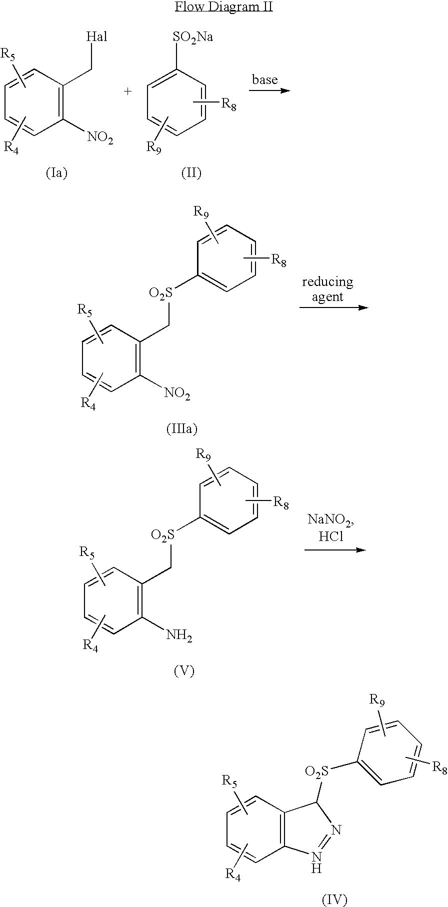

In one embodiment, if R₅ is NR₆R₇, the process comprises preparing a compound of formula Ia by reacting HNR₆R₇ with a compound of formula Ic:

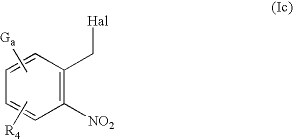

wherein Gₐ is an activating group and R₄ and Hal are as described above for formula Ia.

In another embodiment, the process for the manufacture of a compound of formula IV described hereinabove comprises:

1) reacting a compound of formula Ib

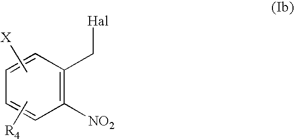

wherein X is either an activating group Gₐ or R₅; R₄ and R₅ are as described hereinabove for formula IV and Hal is Cl, Br or I with a compound of formula II

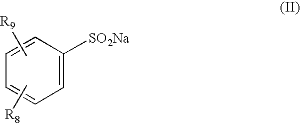

wherein R₈ and R₉ are as described hereinabove for formula IV in the presence of a base to give a compound of formula IIIb

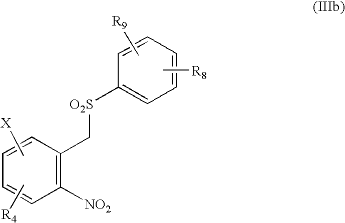

wherein X, R₄, R₈ and R₉ are as described hereinabove;
wherein, where X in formula Ib is R₅ and R₅ is NR₆R₇, the process optionally comprises reacting a compound of formula Ic

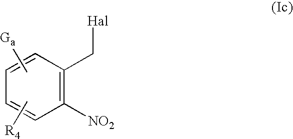

with HNR₆R₇ to give the compound of formula Ib;
with the proviso that if X of formula IIIb is Gₐ and R₅ of formula IV is NR₆R₇, then the process further comprises reacting said compound of formula IIIb with HNR₆R₇ to give the compound of formula IIIb wherein X is R₅ and R₅ is NR₆R₇;

2) reacting said compound of formula IIIb wherein X is R₅ with a reducing agent optionally in the presence of a solvent to give a compound of formula V

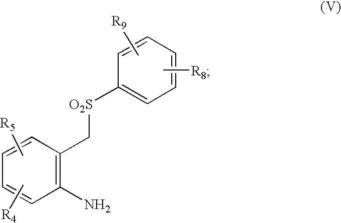

and

3) reacting said compound of formula V with NaNO₂ in the presence of an acid optionally in the presence of a solvent to give the compound of formula IV.

In one embodiment of the above-described processes, activating group Gₐ is Cl, Br or I. Other suitable activating groups include those described above.

In another embodiment of these processes, “NR₆R₇” is an optionally substituted ring of formula VI:

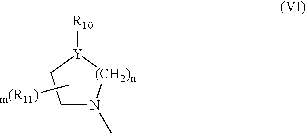

wherein m and n are each independently an integer of 1 to 3;
Y is CH or N with the proviso that if Y is N, n is 2 or 3; and
R₁₀ and each R₁₁ are independently selected from H, C₁-C₆alkyl, C₁-C₆alkoxy, C₁-C₆alkylamino or di-C₁-C₆alkylamino.

Bases suitable for use in the process of the invention include alkali metal carbonates such as K₂CO₃, Na₂CO₃, or the like; alkali metal bicarbonates such as KHCO₃, NaHCO₃, or the like; or any base known to be suitable for use in conventional synthetic procedures, preferably an alkali metal carbonate, more preferably K₂CO₃.

Reducing agents suitable for use in the process of the invention include Sn, HCl; H₂, Pd catalyst, Ra Ni, or the like, preferably Sn, HCl or H₂, Pd catalyst.

Acids suitable for use in Step 3 of the inventive process include mineral acids such as HCl, HBr, or the like, preferably HCl.

Solvents suitable for use in the inventive process include ethers such as tetrahydrofuran; amides such as dimethyl formamide; esters such as ethyl acetate; aromatic hydrocarbons such as toluene; aprotic solvents such as acetonitrile; or the like; preferably tetrahydrofuran.

Among the arylsulfonylindazole compounds of formula IV which may be prepared by the process of the invention are those formula IV compounds wherein R₈ and R₉ are attached to adjacent carbon atoms and are taken together with the atoms to which they are attached to form an optionally substituted 6-membered aromatic aryl ring. In one embodiment, R₈ and R₉ are taken together with the atoms to which they are attached to form a naphthyl ring. Another group of arylsulfonylindazole compounds of formula IV which may be prepared by the process of the invention are those formula IV compounds wherein R₅ is an optionally substituted alkoxy group or an NR₆R₇ group; and R₆ and R₇ are taken together with the atom to which they are attached to form a piperizine ring. A further group of arylsulfonylindazole compounds of formula IV which may be prepared by the process of the invention is those formula IV compounds wherein R₅ is a 3-(dialkyl-amino)propoxy group.

In order to facilitate a further understanding of the invention, the following examples are presented primarily for the purpose of illustrating more specific details thereof. The invention is not to be limited thereby except as defined in the claims.

Unless otherwise noted, all parts are parts by weight. The terms THF and EtOAc designate tetrahydrofuran and ethyl acetate, respectively. The term HPLC designates high performance liquid chromatography.

### EXAMPLES

### Example 1

### Preparation 1-[(5-Fluoro-2-nitrobenzyl)sulfonyl]naphthalene

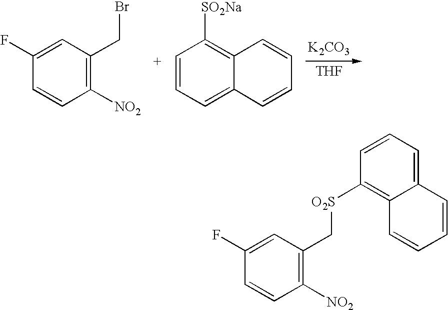

A mixture of THF, 5-fluoro-2-nitrobenzylbromide (2 g, 8.5 mmol), and potassium carbonate (1.2 g, 0085 mol) was stirred under a nitrogen blanket and cooled to 0-5° C. Sodium naphthalene-1-sulfinate (1.8 g, 8.5 mmol) was added to the reaction mixture. The mixture was stirred at 65-67° C. for two hours and the solvent was removed by distillation. The resultant residue was purified by column chromatography (silica gel, 8:2 heptane:EtOAc as elute) to give the title product as a white solid, 2.4 g, 80% yield.

### Example 2

### Preparation of 2-(Phenylsulfonylmethyl)naphthalene

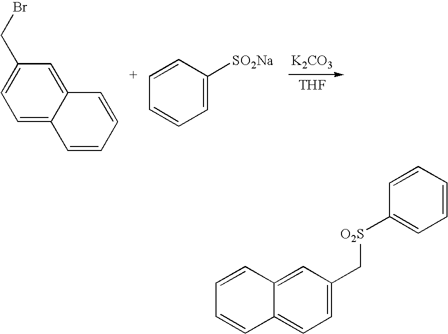

A mixture of THF, K₂CO₃ (2.5 g, 0.018 mol), and sodium benzenesulfinate, (2 g, 0.012 mol) was stirred for 15 minutes under a nitrogen blanket and cooled to 0-5° C. 2-Bromomethylnaphthalene (2.7 g, 0.012 mol) was added to the reaction mixture. The mixture was stirred at 65-67° C. for twelve hours and the solvent was removed by distillation. The resultant solid residue was recrystallized from isopropanol to give the title product, 2.9 g, 86% yield, 88% purity by HPLC.

### Example 3

### Preparation of 3-Methoxy-1-(phenylsulfonylmethyl)benzene

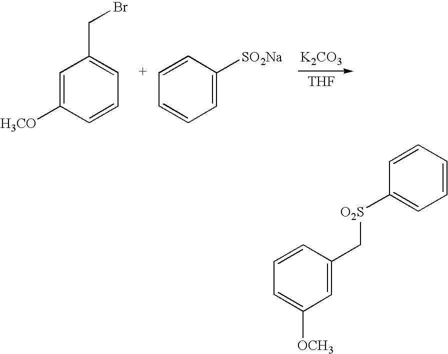

A mixture of THF, K₂CO₃ (2.5 g, 0.018 mol), and sodium benzenesulfinate (2 g, 0.012 mol) was stirred for 15 minutes under a nitrogen blanket and cooled to 0-5° C. 3-Methoxybenzyl bromide (2.6 g, 0.012 mol) was added to the reaction mixture. The mixture was stirred at 65-67° C. for twelve hour, diluted with water and filtered. The filtercake was dried and recrystallized from isopropanol to give the title product, 3.1 g, 99% yield, 88% purity by HPLC.

### Example 4

### Preparation of 4-Nitro-1-(phenylsulfonylmethyl)benzene

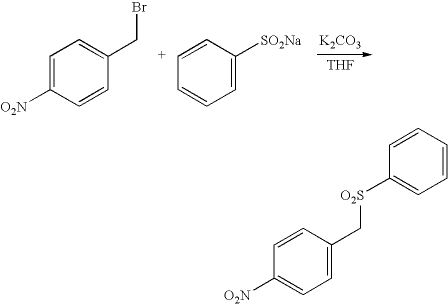

A mixture of THF, K₂CO₃ (2.5 g, 0.018 mol), and sodium benzenesulfinate (2 g, 0.012 mol) was stirred for 15 minutes under a nitrogen blanket and cooled to 0-5° C. 4-Nitrobenzyl bromide (2.6 g, 0.012 mol) was added to the reaction mixture. The mixture was stirred at 65-67° C. for twelve hours, diluted with water and filtered. The filtercake was dried and recrystallized from isopropanol to give the title product, 3.1 g, 93% yield, 87% purity by HPLC.

### Example 5

### Preparation of 4-Chloro-1-[(3-methoxyphenylsulfonyl)methyl]benzene

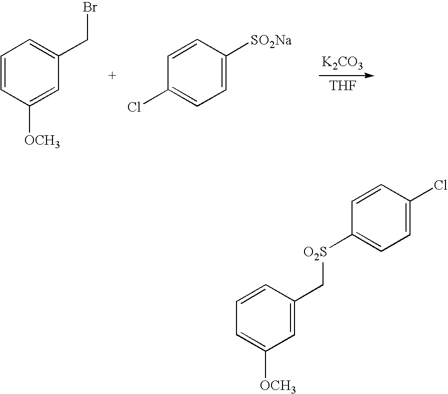

A mixture of THF, K₂CO₃ (0.77 g, 6.0 mmol), and sodium 4-chloro-benzenesulfinate (0.75 g, 3.7 mmol) was stirred for 15 minutes under a nitrogen blanket and cooled to 0-5° C. 3-Methoxybenzyl bromide (0.7 g, 3.7 mmol) was added to the reaction mixture. The mixture was stirred at 65-67° C. for two hours and the solvent was removed by distillation. The resultant solid residue was recrystallized from isopropanol to give the title product, 0.45 g, 64% yield, 99% purity by HPLC.

### Example 6

### Preparation of 2-[(4-chlorophenylsulfonyl)methyl]naphthalene

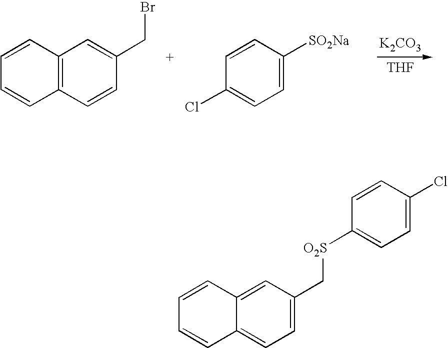

A mixture of THF, K₂CO₃ (0.77 g, 0.006 mol), and sodium 4-chloro-benzenesulfinate (0.75 g, 3.7 mmol) was stirred for 15 minutes under a nitrogen blanket and cooled to 0-5° C. Naphth-2-ylmethylbromide (0.81 g, 3.7 mmol) was added to the reaction mixture. The mixture was stirred at 65-67° C. for two hours and the solvent was removed by distillation. The resultant solid residue was recrystallized from isopropanol to give the title product, 0.30 g, 51% yield, 99% purity by HPLC.

What is claimed is:

1. A process for the manufacture of an arylmethyl sulfonylarene, R₁CH₂SO₂R₂, wherein R₁ and R₂ are each independently an optionally substituted phenyl or naphthyl group which process comprises reacting an arylmethylhalide, R₁CH₂-Hal wherein R₁ is as defined hereinabove and Hal is Cl, Br or I with a sodium arylsulfinate R₂SO₂Na wherein R₂ is as defined hereinabove in the presence of a base optionally in the presence of a solvent.

2. The process according to claim 1 wherein the base is an alkali metal carbonate.

3. The process according to claim 1 wherein the solvent is an ether, an amide, an aromatic hydrocarbon or an aprotic solvent.

4. The process according to claim 3 wherein the solvent is tetrahydrofuran, dimethylformamide, toluene or acetonitrile.

5. The process according to claim 1 wherein the arylmethylhalide is a compound of formula I 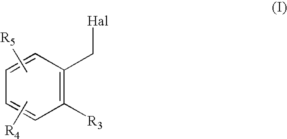
wherein R₃, R₄ and R₅ are each independently H, halogen, NO₂, NR₆R₇, an optionally substituted alkyl or an optionally substituted alkoxy group; R₆ and R₇ are taken together with the atom to which they are attached to form a 5- to 7-membered ring optionally containing an additional heteroatom selected from N, O or S; and Hal is Cl, Br or I.

6. The process according to claim 5 wherein R₃ is NO₂.

7. The process according to claim 6 wherein if R₅ is NR₆R₇, the process comprises preparing a compound of formula I by reacting HNR₆R₇ with a compound of formula Ic: 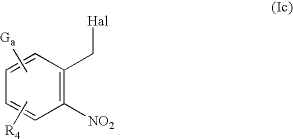
wherein Gₐ is an activating group and R₄ and Hal are as described above for formula I.

8. The process according to claim 7 wherein Gₐ is Cl, Br or I.

9. The process according to claim 7 wherein NR₆R₇ is an optionally substituted ring of formula VI: 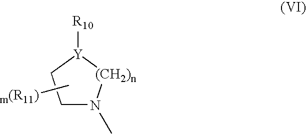
wherein m and n are each independently an integer of 1 to 3;
Y is CH or N with the proviso that if Y is N, n is 2 or 3; and
R₁₀ and each R₁₁ are independently selected from H, C₁-C₆alkyl, C₁-C₆alkoxy, C₁-C₆alkylamino or di-C₁-C₆alkylamino.

10. The process according to claim 5 wherein the sodium arylsulfinate is a compound of formula II 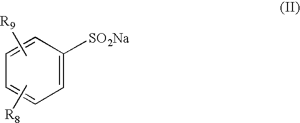
wherein R₈ and R₉ are each independently H, halogen, an optionally substituted alkyl or an optionally substituted alkoxy group or R₈ and R₉ when attached to adjacent carbon atoms are taken together with the atoms to which they are attached to form an optionally substituted 6-membered aromatic aryl ring.

11. The process according to claim 10 wherein R₈ and R₉ are attached to adjacent carbon atoms and are taken together with the atoms to which they are attached to form an optionally substituted 6-membered aromatic aryl ring.

12. The process according to claim 11 wherein R₈ and R₉ are taken together with the atoms to which they are attached to form an optionally substituted naphthyl ring.

13. The process according to claim 11 wherein the base is K₂CO₃.

14. The process according to claim 13 wherein the solvent is tetrahydrofuran.

15. A process for the manufacture of a 3-sulfonylindazole 5-HT6 ligand of formula IV 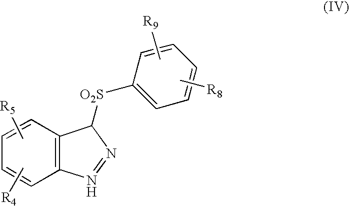
wherein R₄ and R₅ are each independently H, halogen, NO₂, NR₆R₇, an optionally substituted alkyl or an optionally substituted alkoxy group; R₆ and R₇ are taken together with the atom to which they are attached to form a 5- to 7-membered ring optionally containing an additional heteroatom selected from N, O or S; and R₈ and R₉ are each independently H, halogen, an optionally substituted alkyl or an optionally substituted alkoxy group or R₈ and R₉ when attached to adjacent carbon atoms are taken together with the atoms to which they are attached to form an optionally substituted 6-membered aromatic aryl ring
which comprises the following steps:
1) reacting a 2-nitrobenzylhalide of formula Ia 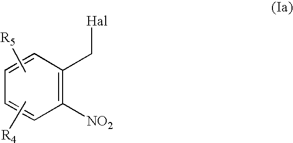
wherein R₄ and R₅ are as described hereinabove for formula IV and Hal is Cl, Br or I with a sodium arylsulfinate of formula II 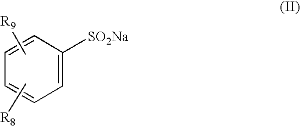
wherein R₈ and R₉ are as described hereinabove for formula IV in the presence of a base to give a compound of formula IIIa 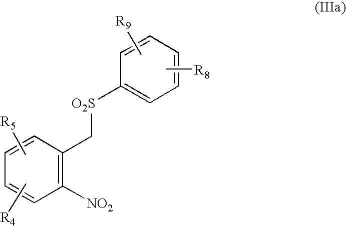
wherein R₄, R₅, R₈ and R₉ are as described hereinabove;
2) reacting said formula IIIa compound with reducing agent to give an amine of formula V 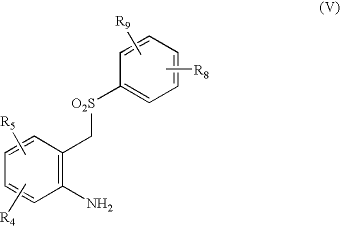
optionally in the presence of a solvent; and
3) reacting said formula V amine with NaNO₂ in the presence of an acid optionally in the presence of a solvent to give the desired 3-sulfonylindazole 5-HT6 ligand of formula IV.

16. The process according to claim 15 wherein if R₅ is NR₆R₇, the process comprises preparing a compound of formula Ia by reacting HNR₆R₇ with a compound of formula Ic: 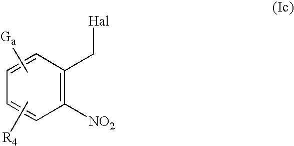
wherein Gₐ is an activating group and R₄ and Hal are as described above for formula Ia.

17. The process according to claim 16 wherein Gₐ is Cl, Br or I.

18. The process according to claim 16 wherein NR₆R₇ is an optionally substituted ring of formula VI: 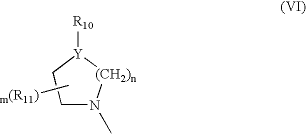
wherein m and n are each independently an integer of 1 to 3;
Y is CH or N with the proviso that if Y is N, n is 2 or 3; and
R₁₀ and each R₁₁ are independently selected from H, C₁-C₆alkyl, C₁-C₆alkoxy, C₁-C₆ alkylamino or di-C₁-C₆alkylamino.

19. The process according to claim 15 wherein the base is an alkali metal carbonate.

20. The process according to claim 15 wherein the reducing agent is Sn, HCl or H₂, Pd catalyst.

21. The process according to claim 15 wherein the acid in Step 3 is HCl.

22. The process according to claim 15 for the manufacture of a formula IV compound wherein R₄ is H and R₅ is an optionally substituted alkoxy group or an NR₆R₇ group; and R₆ and R₇ are taken together with the atom to which they are attached to form a piperizine ring.

23. The process according to claim 22 for the manufacture of a formula IV compound wherein R₈ and R₉ are attached to adjacent carbon atoms and are taken together with the atoms to which they are attached to form an optionally substituted 6-membered aromatic aryl ring.

24. The process according to claim 23 wherein R₈ and R₉ are taken together with the atoms to which they are attached to form an optionally substituted naphthyl ring.

25. The process according to claim 24 for the manufacture of a formula IV compound wherein R₅ is an NR₆R₇ group; and R₆ and R₇ are taken together with the atom to which they are attached to form a piperizine ring.

26. The process according to claim 23 for the manufacture of a formula IV compound wherein R₅ is an optionally substituted alkoxy group.

27. The process according to claim 26 wherein said optionally substituted alkoxy group is a 3-(dialkylamino)propoxy group.

28. The process according to claim 27 wherein said 3-(dialkylamino)-propoxy group is 3-(dimethylamino)propoxy.

29. A process for the manufacture of a compound of formula IV 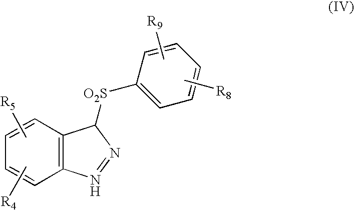
wherein R₄ and R₅ are each independently H, halogen, NO₂, NR₆R₇, an optionally substituted alkyl or an optionally substituted alkoxy group; R₆ and R₇ are taken together with the atom to which they are attached to form a 5- to 7-membered ring optionally containing an additional heteroatom selected from N, O or S; and R₈ and R₉ are each independently H, halogen, an optionally substituted alkyl or an optionally substituted alkoxy group or R₈ and R₉ when attached to adjacent carbon atoms are taken together with the atoms to which they are attached to form an optionally substituted 6-membered aromatic aryl ring which comprises the following steps:
1) reacting a compound of formula Ib 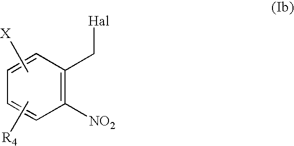
wherein X is either an activating group Gₐ or R₅; R₄ and R₅ are as described hereinabove for formula IV and Hal is Cl, Br or I with a compound of formula II 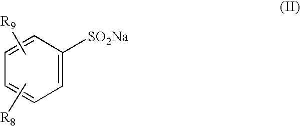
wherein R₈ and R₉ are as described hereinabove for formula IV in the presence of a base to give a compound of formula IIIb 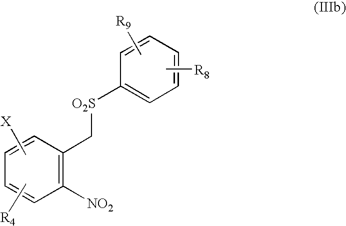
wherein X, R₄, R₈ and R₉ are as described hereinabove,
wherein, where X in formula Ib is R₅ and R₅ is NR₆R₇, the process optionally comprises reacting a compound of formula Ic 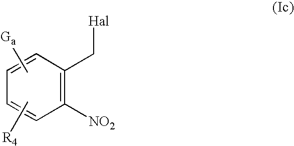
with HNR₆R₇ to give the compound of formula Ib;
with the proviso that if X of formula IIIb is Gₐ and R₅ of formula IV is NR₆R₇, then the process further comprises reacting said compound of formula IIIb with HNR₆R₇ to give the compound of formula IIIb wherein X is R₅ and R₅ is NR₆R₇;
2) reacting said compound of formula IIIb wherein X is R₅ with a reducing agent optionally in the presence of a solvent to give a compound of formula V 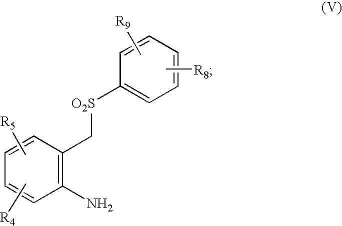
and
3) reacting said compound of formula V with NaNO₂ in the presence of an acid optionally in the presence of a solvent to give the compound of formula IV.

30. The process according to claim 29 wherein Gₐ is Cl, Br or I.

31. The process according to claim 29 wherein NR₆R₇ is an optionally substituted ring of formula VI: 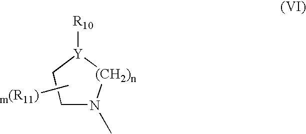
wherein m and n are each independently an integer of 1 to 3;
Y is CH or N with the proviso that if Y is N, n is 2 or 3; and
R₁₀ and each R₁₁ are independently selected from H, C₁-C₆alkyl, C₁-C₆alkoxy, C₁-C₆alkylamino or di-C₁-C₆alkylamino.

32. The process according to claim 29 for the manufacture of a formula IV compound wherein R₄ is H and R₅ is an optionally substituted alkoxy group or an NR₆R₇ group; and R₆ and R₇ are taken together with the atom to which they are attached to form a piperizine ring.

33. The process according to claim 32 for the manufacture of a formula IV compound wherein R₈ and R₉ are attached to adjacent carbon atoms and are taken together with the atoms to which they are attached to form an optionally substituted 6-membered aromatic aryl ring.

34. The process according to claim 33 wherein R₈ and R₉ are taken together with the atoms to which they are attached to form an optionally substituted naphthyl ring.

35. A process for preparing a compound of formula IV 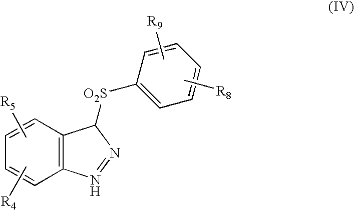
wherein R₄ and R₅ are each independently H, halogen, NO₂, NR₆R₇, an optionally substituted alkyl or an optionally substituted alkoxy group; R₆ and R₇ are taken together with the atom to which they are attached to form a 5- to 7-membered ring optionally containing an additional heteroatom selected from N, O or S; and R₈ and R₉ are each independently H, halogen, an optionally substituted alkyl or an optionally substituted alkoxy group or R₈ and R₉ when attached to adjacent carbon atoms are taken together with the atoms to which they are attached to form an optionally substituted 6-membered aromatic aryl ring
which comprises:
reacting a compound of formula V 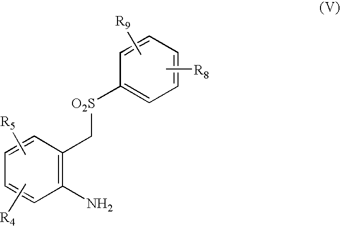
wherein R₄, R₅, R₈ and R₉ are as described above, with NaNO₂ in the presence of an acid optionally in the presence of a solvent to give the compound of formula IV.
